(19) United States
(12) Patent Application Publication
(10) Pub. No.: US 2010/0144675 A1
(43) Pub. Date: Jun. 10, 2010

(54) Regulation of sGC Function by Corrin Derivatives

(76) Inventors: Emil Martin, Houston, TX (US); Ferid Murad, Houston, TX (US)

Correspondence Address: WILLIAMS, MORGAN & AMERSON, 10333 RICHMOND, SUITE 1100, HOUSTON, TX, 77042, US

(21) Appl. No.: 12/515,082

(22) PCT Filed: Jun. 6, 2008
(86) PCT No.: PCT/US08/66008

§ 371 Date: Feb. 19, 2010

Related U.S. Application Data
(60) Provisional application No. 60/942,689, filed on Jun. 8, 2007.

(51) Int. Cl. A61K 31/688; A61K 31/555; A61P 7/02; A61P 9/12; A61P 9/10

(52) U.S. Cl. 514/77; 514/185

(57) ABSTRACT

We disclose a method of activating soluble guanylyl cyclase (sGC) in a patient, comprising administering to the patient a composition comprising a corrin and a pharmaceutically-acceptable carrier. We also disclose a composition, comprising a corrin, an NO-independent sGC activator selected from the group consisting of BAY 41-2272, BAY 58-2667, HMR1766, YC-I, and S3448, and a pharmaceutically-acceptable carrier.

### BACKGROUND OF THE INVENTION

The present invention relates generally to the field of treatment of hypertension, atherosclerosis, thrombosis, or stroke. More particularly, it concerns such treatment by NO-independent activation of soluble guanylyl cyclase (sGC).

### SUMMARY OF THE INVENTION

In one embodiment, the present invention relates to a method of activating soluble guanylyl cyclase (sGC) in a patient comprising administering to the patient a composition comprising a corrin and a pharmaceutically-acceptable carrier.

In one embodiment, the present invention relates to a composition comprising a corrin, an NO-independent sGC activator selected from the group consisting of BAY 41-2272, BAY 58-2667, and HMR1766, and a pharmaceutically-acceptable carrier.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic representation of cGMP-dependent signaling pathways. ANP, atrial natriuretic peptide; BNP, brain natriuretic peptide; cGMP, cyclic guanosine monophosphate; cGMP-PDEs, cyclic guanosine monophosphate phosphodiesterases; CNGC, cyclic nucleotide gated channel; CNP, c-type natriuretic peptide; eNOS, endothelial nitric oxide synthase; GCAP, guanylyl cyclase activating protein; GTP, guanosine triphosphate; iNOS, inducible nitric oxide synthase; nNOS, neuronal nitric oxide synthase; NO, nitric oxide; pGC, membrane-bound guanylyl cyclase; PKGII, cyclic guanosine monophosphate-dependent protein kinase, type II; PKGIα, cyclic guanosine monophosphate-dependent protein kinase, type Iα; PKGIβ, cyclic guanosine monophosphate-dependent protein kinase, type Iβ; sGC, soluble guanylyl cyclase; STa, heat stable enterotoxin.

FIG. 2. Two states of non-NO activated sGC. A. sGC heme iron coordination with His105 is not disrupted by binding of CO to heme iron. B. sGC heme iron coordination with His105 is disrupted by binding of NO to heme iron. C. sGC heme iron coordination with His105 is disrupted by binding of BAY41-2272 to CO-heme. D. sGC heme iron coordination with His105 is disrupted by displacement of sGC heme by protoporphyrin IX (PPIX).

FIG. 3. Structures of tested tetrapyrrole compounds protoporphyrin IX, vitamin B12, Factor B, uroporphyrin I, bilirubin, and nitrite ionophore 1.

FIG. 4. Effect of tetrapyrroles on sGC activity in vitro. A. cGMP production by recombinant human α1/β1 sGC induced by 50 μM factor B, bilirubin, nitrite ionophore 1, uroporphyrin I, or protoporphyrin IX. B. cGMP production by recombinant human α1/β1 sGC induced by 50 μM factor B, bilirubin, nitrite ionophore 1, uroporphyrin I, or protoporphyrin IX alone or in the presence of 1 μM BAY41-2272 or HMR1766. C. cGMP production by recombinant al (31 sGC in response to increasing concentration of protoporphyrin, factor B, vitamin B12 and vitamin B12a. D. cGMP production by recombinant human al/131 sGC induced by various doses of factor B at various doses of BAY41-2272. E. cGMP production by recombinant human α1/β1 sGC induced by various doses of factor B at various doses of BAY41-2272.

FIG. 5. Synergistic effect of corrin derivatives and BAY41-2272 on relaxation of rat aortic rings. The measurements were performed in the presence of corrin derivatives±BAY41-2272 as described in the text. Data are average of two aortic rings per each dose-response curve. Arrow indicates the shift in the effective dose and increased response.

FIG. 6. Effect of factor B and BAY41-2272 on mean arterial pressure (MAP) in instrumented anesthetized rats. Indicated amount of drugs were delivered as a 500 μl bolus through a catheter into the abdominal aorta and MAP were recorded as described in section D1.2. “Contr”—500 μl of saline,

FIG. 7. Changes in cGMP levels in rat aortic rings. Aortic rings were incubated with saline or saline with factor B in the presence or absence of BAY 41-2272 for 5 minutes at 37° C. cGMP was extracted and quantified.

FIG. 8. Other tetrapyrrole activators.

### DESCRIPTION OF ILLUSTRATIVE EMBODIMENTS

In one embodiment, the present invention relates to a method of activating soluble guanylyl cyclase (sGC) in a patient comprising administering to the patient a composition comprising a corrin and a pharmaceutically-acceptable carrier.

A corrin, as used herein, refers to a compound having the general formula (I)

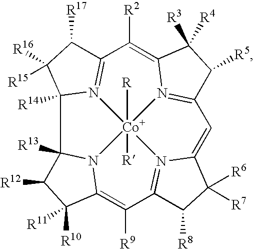

wherein R is selected from the group consisting of —CN, —N³—, —OH, —CₓH₂ₓ₊₁ wherein x is an integer from 1 to about 10, inclusive, and -deoxyadenosyl;.

R¹ is selected from the group consisting of —CN, —N³⁻, —OH, —CₓH₂ₓ₊₁ wherein x is an integer from 1 to about 10, inclusive, -deoxyadenosyl, and dimethylbenzimidazolyl;

R² is selected from the group consisting of —H and —CₓH₂ₓ₊₁ wherein x is an integer from 1 to about 10, inclusive;

R³ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;

R⁴ is selected from the group consisting of —H and —CₓH₂ₓ₊₁ wherein x is an integer from 1 to about 10, inclusive;

R⁵ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;

R⁶ is selected from the group consisting of —H and —CₓH₂ₓ₊₁ wherein x is an integer from 1 to about 10, inclusive;

R⁷ is selected from the group consisting of —H and —CₓH₂ₓ₊₁ wherein x is an integer from 1 to about 10, inclusive;

R⁸ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;

R⁹ is selected from the group consisting of —H and —CₓH₂ₓ₊₁ wherein x is an integer from 1 to about 10, inclusive;

R¹⁰ is selected from the group consisting of —H and —CₓH₂ₓ₊₁ wherein x is an integer from 1 to about 10, inclusive;

R¹¹ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, —C_{y}H_{2y}CONH₂ wherein y is an integer from 1 to about 9, inclusive, —CH₂CH₂CONHCH₂CH(OH)CH₃, and —C_{y}H_{2y}CONHCH₂CHOP(OC₅O₃H₈)O₂ wherein y is an integer from 1 to about 9, inclusive, and (C₅O₃H₈) forms a ribose group;

R¹² is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;

R¹³ is selected from the group consisting of —H and —CₓH₂ₓ₊₁ wherein x is an integer from 1 to about 10, inclusive;

R¹⁴ is selected from the group consisting of —H and —CₓH₂ₓ₊₁ wherein x is an integer from 1 to about 10, inclusive;

R¹⁵ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;

R¹⁶ is selected from the group consisting of —H and —CₓH₂ₓ₊₁ wherein x is an integer from 1 to about 10, inclusive; and

R¹⁷ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive.

The ribose of R¹¹ can be covalently linked with the dimethylbenzimidazolyl of R¹.

In one embodiment, the corrin is selected from the group consisting of dicyanocobinamide (Factor B), having formula I wherein R and R¹ are —CN; R², R³, R⁶, R⁷, R⁹, R¹⁰, R¹⁴, and R¹⁶ are —CH₃; R¹³ is —H; R⁴, R¹², and R¹⁵ are —CH₂CONH₂; R⁵, R⁸, and R¹⁷ are —CH₂CH₂CONH₂; and R¹¹ is —CH₂CH₂CONHCH₂CH(OH)CH₃; cyanocobalamin (vitamin B₁₂), having structure II,

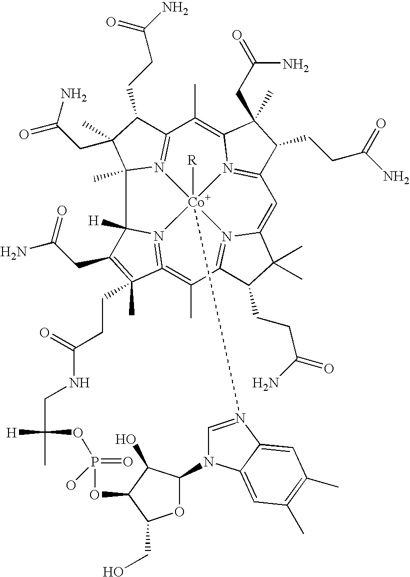

wherein R is —CN; methylcobalamin (MeB₁₂), having structure II, wherein R is —CH₃; 5-deoxyadenosylcobalamin (AdoB₁₂), having structure II, wherein R is -deoxyadenosyl; and hydroxycobalamin (HOB₁₂), having structure II, wherein R is —OH.

The pharmaceutically-acceptable carrier can be any compound in which the corrin can be mixed or dissolved without significant (i.e., less than 10%) chemical decomposition within 30 days after mixing or dissolution, and which is known for use in the administration of pharmaceutical compounds to a patient. Parameters which may considered to determine the pharmaceutical acceptability of a carrier can include, but are not limited to, the toxicity of the pharmaceutically-acceptable carrier, the interaction between the compound having structure I and the pharmaceutically-acceptable carrier, the approval by a regulatory body of the pharmaceutically-acceptable carrier for use in medicaments, or two or more of the foregoing, among others.

The pharmaceutically-acceptable carrier can be any material or plurality of materials which can form a composition with the compound having structure I. The particular carrier can be selected by the skilled artisan in view of the intended use of the composition and the properties of the compound having structure I, among other parameters apparent in light of the present disclosure.

Non-limiting examples of particular carriers and particular compositions follow.

In one embodiment, the pharmaceutically-acceptable carrier is water, and the composition is an aqueous solution comprising water and the compound having structure I. An example of pharmaceutically-acceptable carrier is an aqueous saline solution. The composition can further comprise solutes, such as salts, acids, bases, or mixtures thereof, among others. The composition can also comprise a surfactant, an emulsifier, or another compound capable of improving the solubility of the compound having structure I in water.

In one embodiment, the pharmaceutically-acceptable carrier is a polar organic solvent, and the composition is a polar organic solution comprising the polar organic solvent and the compound having structure I. “Polar” has its standard meaning in the chemical arts of describing a molecule that has a permanent electric dipole. A polar molecule can but need not have one or more positive, negative, or both charges. Examples of polar organic solvents include, but are not limited to, methanol, ethanol, formate, acrylate, or mixtures thereof, among others. The composition can further comprise solutes, such as salts, among others. The composition can also comprise a surfactant, an emulsifier, or another compound capable of improving the solubility of the compound having structure I in the polar organic solvent.

In one embodiment, the pharmaceutically-acceptable carrier is an apolar organic solvent, and the composition is an apolar organic solution comprising the apolar organic solvent and the compound having structure I. “Apolar” has its standard meaning in the chemical arts of describing a molecule that does not have a permanent electric dipole. The composition can further comprise solutes, such as apolar molecules, among others. The composition can also comprise a surfactant, an emulsifier, or another compound capable of improving the solubility of the compound having structure I in the apolar organic solvent. In one embodiment, the composition is a water-in-oil emulsion, wherein the compound having structure I is dissolved in water and water is emulsified into a continuous phase comprising one or more apolar organic solvents.

In one embodiment, the pharmaceutically-acceptable carrier is a solid or semisolid carrier, and the composition is a solid or semisolid matrix in or over which the compound having structure I is dispersed. Examples of components of solid carriers include, but are not limited to, sucrose, gelatin, gum arabic, lactose, methylcellulose, cellulose, starch, magnesium stearate, talc, petroleum jelly, or mixtures thereof, among others. The dispersal of the compound having structure I can be homogeneous (i.e., the distribution of the compound having structure I can be invariant across all regions of the composition) or heterogeneous (i.e., the distribution of the compound having structure I can vary at different regions of the composition). The composition can further comprise other materials, such as flavorants, preservatives, or stabilizers, among others.

In one embodiment, the pharmaceutically-acceptable carrier is a gas, and the composition can be a gaseous suspension of the compound having structure I in the gas, either at ambient pressure or non-ambient pressure. Examples of the gas include, but are not limited to, air, oxygen, nitrogen, or mixtures thereof, among others.

Other carriers will be apparent to the skilled artisan having the benefit of the present disclosure.

In one embodiment, the composition further comprises an NO-independent sGC activator, i.e., a compound that stimulates sGC activity but does not stimulate NO production by a nitric oxide synthase or mimic the interaction of NO with the sGC. In one embodiment, the NO-independent sGC activator is selected from the group consisting of BAY 41-2272, BAY 58-2667, and HMR1766. BAY 41-2272 has structure III:

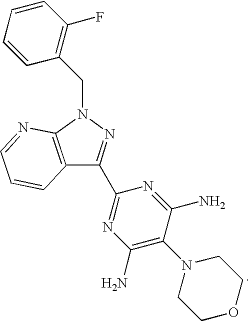

BAY 58-2667 has structure IV:

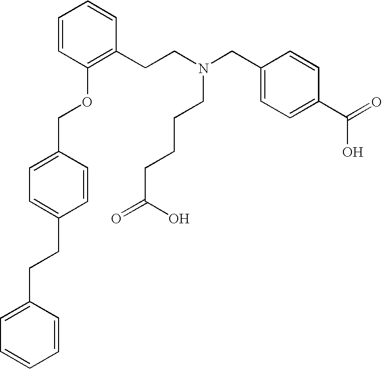

HMR1766 has structure V:

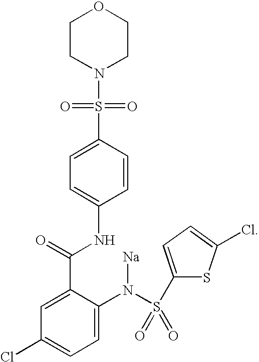

In another embodiment, the NO-independent sGC activator is selected from the group consisting of YC-1 and S3448. YC-1 has structure VI:

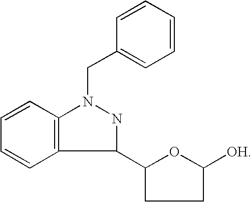

S3448 has structure VII:

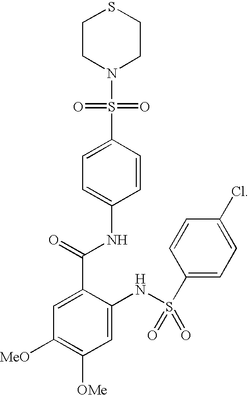

In one embodiment, the present invention relates to a composition comprising a corrin, an NO-independent sGC activator selected from the group consisting of BAY 41-2272, BAY 58-2667, HMR1766, YC-1, and S3448, and a pharmaceutically-acceptable carrier. The corrin, the NO-independent sGC activator, and the pharmaceutically-acceptable carrier can be as described above. In one embodiment, the corrin is selected from the group consisting of dicyanocobinamide (Factor B), cyanocobalamin (vitamin B₁₂), methylcobalamin (MeB₁₂), and 5-deoxyadenosylcobalamin (AdoB₁₂).

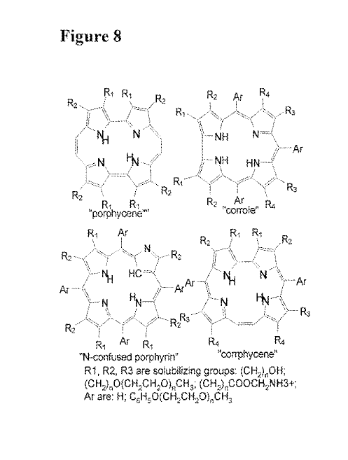

In one embodiment, the present invention relates to a method of activating soluble guanylyl cyclase (sGC) in a patient comprising administering to the patient a composition comprising a moiety selected from the group consisting of a porphycene, a corrole, an N-confused porphyrin, a corrphycene, and non-cobalt corrins, and a pharmaceutically-acceptable carrier. The structures of porphycenes, corroles, N-confused porphyrins, and corrphycenes are given in FIG. 8, and have a central metal atom (not shown) selected from the group consisting of Co, Fe, Ni, Zn, Cu, and Mn. A non-cobalt corrin has a structure similar to structure I, differing in that the cobalt atom is replaced with a metal atom selected from the group consisting of Fe, Ni, Zn, Cu, and Mn.

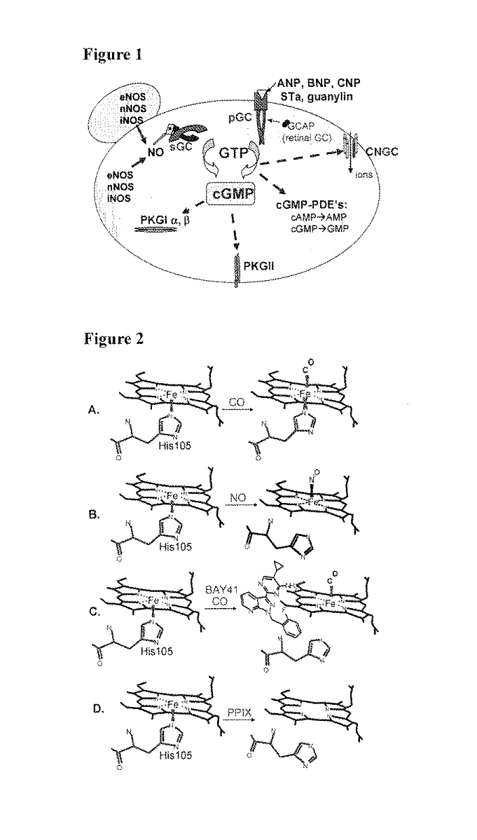

Cyclic 3′, 5′-guanosine monophosphate (cGMP) is an important intracellular messenger involved in various aspects of cell physiology and tissue homeostasis. This cGMP messenger controls diverse processes, such as vascular and other smooth muscle relaxation, neurotransmission, hormonal secretion and platelet aggregation¹, ². This diversity of physiological functions is achieved through recruitment of cyclic nucleotide phosphodiesterases (PDE's), cGMP-gated ion channels, and cGMP-dependent protein kinases (PKGs) (FIG. 1). Intracellular cGMP level is controlled by a family of guanylyl cyclases, which can be membrane-bound (pGC, FIG. 1) or cytosolic (sGC in FIG. 1). Binding of extracellular peptide ligands (ANP, BNP, etc) or intracellular activating proteins, such as GCAP, regulate the function of membrane-bound GC. Function of the cytosolic enzyme, called soluble guanylyl cyclase, is activated after nitric oxide (NO) produced either intracellular or extracellular by NO synthases bind to sGC heme.

In summary, activation of sGC enzyme increases intracellular levels of cGMP, which induces several cGMP-dependent downstream targets affecting protein phosphorylation, intracellular balance of cyclic nucleotides and influx of ions.

The sGC enzyme is a heterodimer composed of one larger a subunit (˜82 kDa for human enzyme) and one smaller β subunit (76 kDa for human enzyme)³ and contains one type IX heme moiety per molecule of sGC². Although two isoforms of each subunit have been identified—α1, α2, β1, β2 in various species ranging from worms and insects to mammals⁴, only α1/β1 and α2/β1 heterodimers have been found so far in vivo¹. The α1/β1 enzyme is the most abundant and is ubiquitously expressed.

Based on the rate of cGMP production activated sGC exists in two states. In the low-output state the enzyme produces only slightly elevated amounts of cGMP as compared to the resting enzyme. E.g. carbon monoxide (CO)-stimulated sGC produces only 3-4 time more cGMP that the resting enzyme. On the contrary the NO-stimulated enzyme is in the high-output state since it produces cGMP with >100 fold increased rate. The iron ion from the heme moiety maintains a coordinating bond with the histidine 105 residue of the β subunit when the enzyme is in the resting state. In the low-output state this coordinating bond is maintained (FIG. 2A)^{5,6}, while transition to the high-output state of NO-sGC complex coincide with the cleavage of this His105-heme bond (FIG. 2B)^{7,8}. Low-output CO-sGC complex can be converted into the high-output state by treatment with allosteric regulator BAY41-2272 (FIG. 2C)⁹. Resonance Raman measurements of the CO-sGC complex also confirmed that the transition into high cGMP output state is associated with the cleavage of coordinating bond upon addition of BAY41-2272 (BAY41-2272)¹⁰. Another mechanism of high-output sGC activation was described early on by Ignarro and co-workers¹¹. They reported that protoporphyrin IX (PPIX) activated efficiently the sGC enzyme. It was proposed that PPIX-treated enzyme form is converted into a state similar to the NO-activated sGC. Later it has been proposed that the heme is replaced with PPIX, which is not able to support the His105-heme bond (FIG. 2D). Thus, the concept that His105-heme bond cleavage is required for sGC activation was conceived¹².

Recently this concept found confirmation in the discovery of heme-independent activators of sGC. Two compounds, BAY58-2667 and HM1766, were identified in the drug discovery program of Bayer¹³ and Sanofi-Aventis¹⁴, respectively. Although these two substances are not chemically related, they have similar effects of sGC activation. These heme-independent activators are not effective when the enzyme contains the full complement of heme. However, direct depletion of heme by detergents, or oxidation of heme iron by sGC inhibitor ODQ, significantly enhances sGC activation by BAY58-2667¹³ or HM1766¹⁴. Spectral studies demonstrated that these activators decrease the intensity of the UV-Vis spectra of the Soret region in a dose-dependent manner. This clearly indicates that the mechanism of activation is the replacement of the heme moiety with BAY58-2667 or HM1766 compounds. Moreover, mutations of residues responsible for interaction with the heme also prevented activation of the enzyme by BAY58-2667^{15,16}.

In summary, ligand-induced disruption of the coordinating bond between the heme's iron and His 105 residue of sGC β subunit, or replacement of heme moiety with PPIX or heme-independent activators converts sGC into a high cGMP output state.

SGC is an important therapeutic target and regulation of sGC activity is of significant cardiovascular importance. NO-dependent increase of intracellular cGMP mediated by sGC induces relaxation of vascular smooth muscle cells, resulting in vasodilatation¹⁷. Mice lacking the a subunit develop hypertension¹⁸, suggesting that sGC is a primary target for hypertension treatment. Such NO-donors as glyceryl trinitrate, isosorbide dinitrate, isosorbide-5-mononitrate are used to unload the heart in cases of angina pectoris, myocardial infarction, congestive heart failure and hypertension¹⁹. However, patients treated with these nitrovasodilators often develop tolerance, which limit their efficiency²⁰. Moreover, activation of sGC in platelets reduces platelet aggregation and leukocyte adherence and is beneficial in cases of atherosclerosis, thrombosis and stroke²¹. However, nitrovasodilators show little effect on platelet aggregation²². Thus, finding alternative pharmacologically effective methods of sGC activation or improving the existing methods are of great clinical interest.

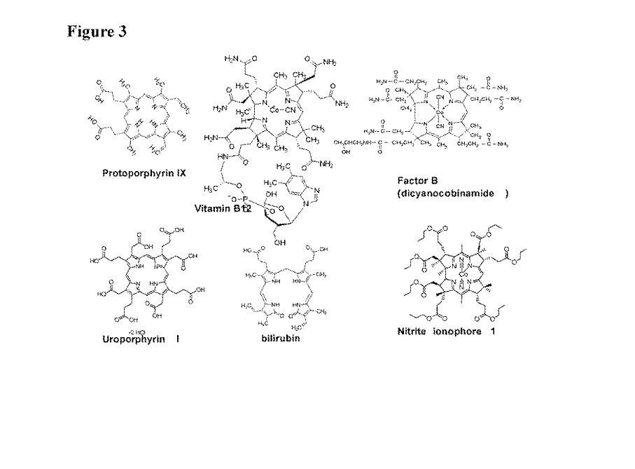

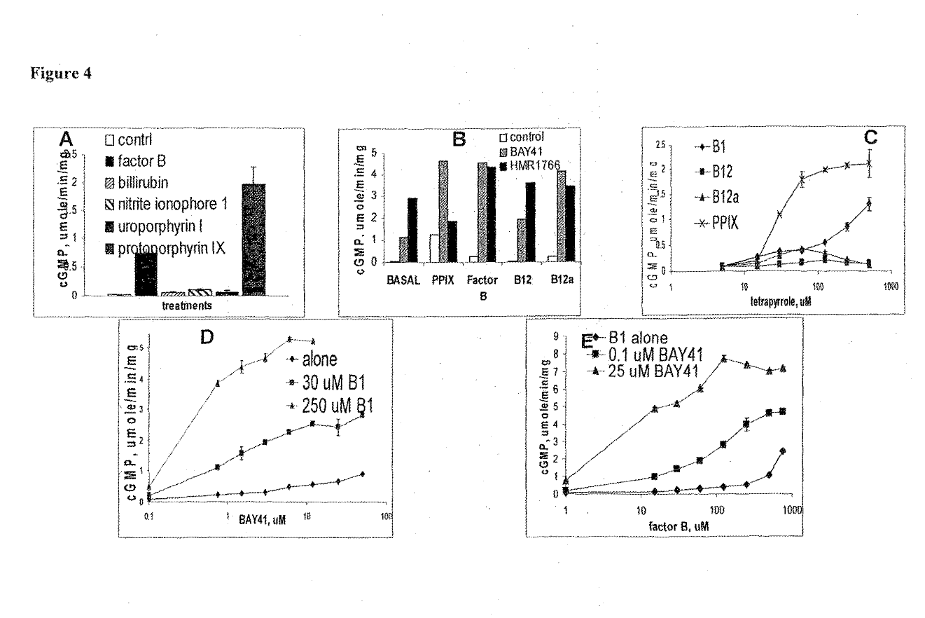

Activation of purified sGC by various tetrapyrroles. Since replacement of SGC heme moiety by PPIX, BAY58-2667, or HMR1766 compounds results in significant activation of the enzyme, we investigated whether other compounds may have similar effects. Methods of recombinant expression and purification of human α1/β11 isoform of sGC have been developed^{23, 24}. Measurements of enzymatic activity and multiple spectral studies performed using this recombinant enzyme^{10, 23, 25} indicate that these recombinant preparations have identical properties as the native enzyme. We tested a series of tetrapyrrole compounds (FIG. 3), which are structurally related to PPIX. Similar to PPIX, majority of the tested tetrapyrroles contain four pyrrole rings connected to form a microcycle. In the case of billirubin, however, the microcycle is interrupted. Of tested tetrapyrroles at 50 μM, besides PPIX, only factor B (fB), the precursor of vitamin B12, significantly activated sGC (FIG. 4A). We then compared fB with vitamin B12 (cyanocobalamine) or its derivative B12a (aquacobalamine) and PPIX, all at 50 μM. We found that fB is an effective activator of sGC, especially when it is facilitated by 1 μM allosteric activators BAY41-2272 or heme-independent activator HMR1766 (FIG. 4B). HMR1766 was kindly provided by Sanofi-Avantis (see a letter attached). Together with BAY41-2272, fB induced the enzyme to a similar extent as the PPIX/BAY41-2272 combination, while in combination with HMR1766 it was even more effective than PPIX/HMR1766 (FIG. 4B). Measurement of the dose response of several corrins indicates that sGC activation by vitamin B12 and vitamin B12a is maximal at 50 μM, while activation by factor B did not reach maximum in the tested range of concentrations. The combination of BAY41-2272 and fB not only synergistically enhanced the enzyme, but also shifted the EC50 to lower concentrations for both BAY41-2272 (FIG. 4D) and factor B (FIG. 4E). These data allowed us to conclude that combined treatment with factor B and BAY41-2272 may be an effective approach to induce the high-output state of sGC and may be an effective method of sGC regulation in vivo.

FB-dependent relaxation of isolated rat aorta. In addition to in vitro analysis of sGC activity, we tested whether fB and vitamin B12 derivatives will induce relaxation of preconstricted rat aortic rings. The descending thoracic aorta was excised from freshly thoracic aorta was excised from freshly sacrificed Sprague Dawley rats, cleaned of adherent tissue and cut into 3-4 mm rings. Each ring was suspended between two wires hooks and mounted under 1 mN of passive tension in 4 ml organ chambers filled with Krebs' solution at 37° C., pH 7.4 and continuously aerated with 95% O₂/5% CO₂. The changes in isometric tension were measured by a force transducer (Grass Instruments, USA) connected to a PowerLab 400™ data-acquisition system (ADInstruments, Colorado Springs, Mass., USA). Ring preparations were equilibrated for 1 h before the start of the experiments. After the equilibration period, rings were preconstricted with 1 μM phenylephrine and maximal contractile response is determined. After maximal constriction the agonists (vitamin B12, methyl-B12, fB, BAY41-2272) were added cumulatively and changes in isometric tension determined. Data plotted as percentage of the maximal contraction are presented in FIG. 5. These measurements demonstrated that all corrin derivatives (vitamin B12, methyl-B12, and fB) can induce vascular relaxation. In the presence of a low dose of BAY41-2272 (0.1 μM) EC50 of fB decreased while the response increased. Similar effects were observed with vitamin B12 and methyl-B12 (data not shown). This synergistic enhancement by BAY41-2272 of corrin-mediated aortic ring relaxation suggests that this phenomenon is sGC-dependent. Essentially similar response was observed in endothelium denudated aortic rings, indicating that this is relaxation occurs NO-independently (data not shown). In conclusion, our data indicate that corrin derivatives induce relaxation of aortic rings NO-independently and this relaxation can be synergistically enhanced by sGC allosteric activator BAY41-2272.

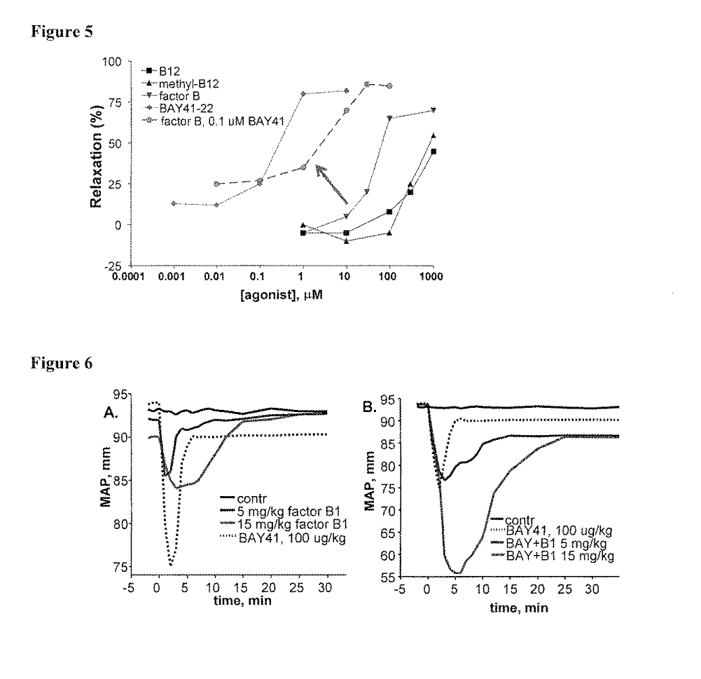

Effect of factor B and BAY41-2272 on blood pressure of anesthetized rats. We also measured the effect of bolus application of fB on changes in hemodynamic parameters in anesthetized rats. Sprague Dawley rats were surgically implanted with a 10-MHz pulsed Doppler flow and a displacement probe around the ascending aorta and sutured to the left ventricular wall, respectively. A Tygon catheter was placed into the abdominal aorta via the femoral artery. After recovery the animals were anesthetized with halothane, intubated, and ventilated under isothermic conditions. Changes in systolic, diastolic, mean arterial pressures and cardiac output were recorded and processed with a multichannel-pulsed Doppler flow/dimension system and digitized (A/D converter). Before experimental treatments with fB and/or BAY41-2272 the animals were treated with a bolus of 30 μg/kg of sodium nitroprusside to confirm the integrity of the catheter and delivery system and the responsiveness of animals to drugs. After the baseline was established a 500 μl bolus of fB at 5 and 15 mg/ml or 100 μg/kg of BAY41-2272 (Alexis, San Diego, Calif., USA) was delivered and the changes in blood pressure (FIG. 6A). and cardiac output were recorded (data not shown). Transient decreased in blood pressure coincided with increased cardiac output, strongly suggesting that changes in blood pressure were due to increased vasodilatation. Corroborating previous reports⁹ we observed that after administration of BAY41-2272 animals stabilize their MAP at lower values, which are maintained for at least 30 minutes. When added together with 100 μg/kg of BAY41-2272, fB not only enhanced the amplitude of BAY41-2272-induced drop in blood pressure, but also significantly prolonged the time of acutely decreased blood pressure (FIG. 6B). Most importantly, the baseline to which the MAP was restored in animals with combined fB/BAY41-2272 treatment was even lower and more prolonged that after administration of BAY41-2272 alone (FIG. 6B). These data suggest that factor B alone or in combination with allosteric activators of sGC may be an efficient and inexpensive way to manage blood pressure in hypertensive animals.

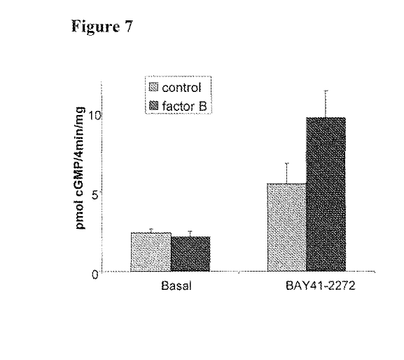

Changes in cGMP levels in isolated rat aorta treated with factor B and BAY 41-2272. In order to confirm that relaxation of blood vessels and decreased blood pressure in response to factor B is dependent on sGC activity we measured cGMP levels in treated aortic rings. As demonstrated in FIG. 7, levels of cGMP indeed increased after 5 minutes incubation with factor B in combination with BAY 41-2272.

Modification of the corrin compounds through changes of chelated metal and/or coordinated ligands. Removal of cobalt ion from the fB is not possible without disintegration of the corrin ring⁴⁵, while direct substitution of cyanide by other ligands is not very effective. Thus, the corrole, precursor of corrin, can be used to insert the metals ion in the core of the microcycle followed by addition of ligand. To insert the metal ion the following experimental steps described previously⁴⁶ can be performed: (1) 60 mg of corrole dissolved in 20 ml of pyridine can be heated to reflux in a rotary evaporator (Buchi Labortechnik AG, Germany) (2) A small amount of metal salt (Ni₂SO₄, or CuSO₄, or ZnNO₃), can be added to the hot solution to start the insertion reaction. The reaction progress can be monitored by TLC examination on silica plates with n-hexane/CH₂Cl₂=2:1 as solvent carrier. The spots can be visualized by UV lamp. After metal insertion, corrole will not only change color, but will also lose fluorescence. In case the reaction is not completed, additional metal salt can be added and incubation continued until at least 80% of corrole is converted into metal containing corrin. (3) Once the reaction is complete and the solvent is evaporated, the solid material can be dissolved in CH₂Cl₂ and filtered to remove inorganic salts. (4) A 3-5 g portion of silica gel can be added to the filtrate, and the solvent can be evaporated. Purification of the product can be performed by column chromatography on silica gel, with a 22:3 mixture of n-hexane and CH₂Cl₂ as described previously⁴⁶. As mentioned above, the reaction produce can be identified by change in color and decreased fluorescence. To add the ligand groups, excess of sodium azide or cyanide can be added after step (3) and purified through same methods. Modified corrin compounds can be tested directly on purified sGC and on intact cell.

Peripheral modifications of the corrole can be performed as shown in FIG. 8. Based on the results from the previous step, the appropriate metal ion and ligand can be attached to these new derivatives.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Lucas, K. A.; Pitari, G. M.; Kazerounian, S.; Ruiz-Stewart, I.; Park, J.; Schulz, S.; Chepenik, K. P.; Waldman, S. A., Guanylyl cyclases and signaling by cyclic GMP. Pharmacol Rev 2000, 52, (3), 375-414.
2. Denninger, J. W.; Marietta, M. A., Guanylate cyclase and the NO/cGMP signaling pathway. Biochim Biophys Acta 1999, 1411, (2-3), 334-50.
3. Giuili, G.; Scholl, U.; Bulle, F.; Guellaen, G., Molecular cloning of the cDNAs coding for the two subunits of soluble guanylyl cyclase from human brain. FEBS Lett 1992, 304, (1), 83-8.
4. Wedel, B.; Garbers, D., The guanylyl cyclase family at Y2K. Annu Rev Physiol 2001, 63, 215-33.
5. Burstyn, J. N.; Yu, A. E.; Dierks, E. A.; Hawkins, B. K.; Dawson, J. H., Studies of the heme coordination and ligand binding properties of soluble guanylyl cyclase (sGC): characterization of Fe(II) sGC and Fe(II) sGC(CO) by electronic absorption and magnetic circular dichroism spectroscopies and failure of CO to activate the enzyme. Biochemistry 1995, 34, (17), 5896-903.
6. Stone, J. R.; Marietta, M. A., Soluble guanylate cyclase from bovine lung: activation with nitric oxide and carbon monoxide and spectral characterization of the ferrous and ferric states. Biochemistry 1994, 33, (18), 5636-40.
7. Zhao, Y.; Brandish, P. E.; Ballou, D. P.; Marietta, M. A., A molecular basis for nitric oxide sensing by soluble guanylate cyclase. Proc Natl Acad Sci USA 1999, 96, (26), 14753-8.
8. Ballou, D. P.; Zhao, Y.; Brandish, P. E.; Marietta, M. A., Revisiting the kinetics of nitric oxide (NO) binding to soluble guanylate cyclase: the simple NO-binding model is incorrect. Proc Natl Acad Sci USA 2002, 99, (19), 12097-101.
9. Stasch, J. P.; Becker, E. M.; Alonso-Alija, C.; Apeler, H.; Dembowsky, K.; Feurer, A.; Gerzer, R.; Minuth, T.; Perzborn, E.; Pleiss, U.; Schroder, H.; Schroeder, W.; Stahl, E.; Steinke, W.; Straub, A.; Schramm, M., NO-independent regulatory site on soluble guanylate cyclase. Nature 2001, 410, (6825), 212-5.
10. Martin, E.; Czarnecki, K.; Jayaraman, V.; Murad, F.; Kincaid, J., Resonance Raman and infrared spectroscopic studies of high-output forms of human soluble guanylyl cyclase. J Am Chem Soc 2005, 127, (13), 4625-31.
11. Ignarro, L. J.; Wood, K. S.; Wolin, M. S., Activation of purified soluble guanylate cyclase by protoporphyrin IX. Proc Natl Acad Sci USA 1982, 79, (9), 2870-3.
12. Sharma, V. S.; Magde, D., Activation of soluble guanylate cyclase by carbon monoxide and nitric oxide: a mechanistic model. Methods 1999, 19, (4), 494-505.
13. Stasch, J. P.; Schmidt, P.; Alonso-Alija, C.; Apeler, H.; Dembowsky, K.; Haerter, M.; Heil, M.; Minuth, T.; Perzborn, E.; Pleiss, U.; Schramm, M.; Schroeder, W.; Schroder, H.; Stahl, E.; Steinke, W.; Wunder, F., NO- and haem-independent activation of soluble guanylyl cyclase: molecular basis and cardiovascular implications of a new pharmacological principle. Br J Pharmacol 2002, 136, (5), 773-83.
14. Schindler, U.; Strobel, H.; Schonafinger, K.; Linz, W.; Lohn, M.; Martorana, P. A.; Rutten, H.; Schindler, P. W.; Busch, A. E.; Sohn, M.; Topfer, A.; Pistorius, A.; Jannek, C.; Mulsch, A., Biochemistry and pharmacology of novel anthranilic acid derivatives activating heme-oxidized soluble guanylyl cyclase. Mol Pharmacol 2006, 69, (4), 1260-8.
15. Schmidt, P. M.; Rothkegel, C.; Wunder, F.; Schroder, H.; Stasch, J. P., Residues stabilizing the heme moiety of the nitric oxide sensor soluble guanylate cyclase. Eur J Pharmacol 2005, 513, (1-2), 67-74.
16. Schmidt, P. M.; Schramm, M.; Schroder, H.; Wunder, F.; Stasch, J. P., Identification of Residues Crucially Involved in the Binding of the Heme Moiety of Soluble Guanylate Cyclase. J Biol Chem 2004, 279, (4), 3025-3032.
17. Ignarro, L. J., Nitric oxide: a unique endogenous signaling molecule in vascular biology. Biosci Rep 1999, 19, (2), 51-71.
18. Mergia, E.; Friebe, A.; Dangel, O.; Russwurm, M.; Koesling, D., Spare guanylyl cyclase NO receptors ensure high NO sensitivity in the vascular system. J Clin Invest 2006, 116, (6), 1731-7.
19. Abrams, J., Beneficial actions of nitrates in cardiovascular disease. Am J Cardiol 1996, 77, (13), 31C-7C.
20. Torfgard, K. E.; Ahlner, J., Mechanisms of action of nitrates. Cardiovasc Drugs Ther 1994, 8, (5), 701-17.
21. Tulis, D. A., Salutary properties of YC-1 in the cardiovascular and hematological systems. Curr Med Chem Cardiovasc Hematol Agents 2004, 2, (4), 343-59.
22. Rao, G. H.; Rao, A. T., Pharmacology of platelet activation-inhibitory drugs. Indian J Physiol Pharmacol 1994, 38, (2), 69-84.
23. Martin, E.; Berka, V.; Tsai, A. L.; Murad, F., Soluble Guanylyl Cyclase: The Nitric Oxide Receptor. Methods Enzymol 2005, 396PE, 478-492.
24. Martin, E.; Sharina, I.; Kots, A.; Murad, F., A constitutively activated mutant of human soluble guanylyl cyclase (sGC): implication for the mechanism of sGC activation. Proc Natl Acad Sci USA 2003, 100, (16), 9208-13.
25. Martin, E.; Berka, V.; Bogatenkova, E.; Murad, F.; Tsai, A. L., Ligand selectivity of soluble guanylyl cyclase: effect of the hydrogen-bonding tyrosine in the distal heme pocket on binding of oxygen, nitric oxide, and carbon monoxide. J Biol Chem 2006, 281, (38), 27836-45.
26. Nikolaev, V. O.; Gambaryan, S.; Lohse, M. J., Fluorescent sensors for rapid monitoring of intracellular cGMP. Nat Methods 2006, 3, (1), 23-5.
27. Doursout, M.-F.; Kashimoto, S.; Chelly, J., Cardiac function in the diabetic heart. In The Diabetic Heart, ed.; Nagano M, D. M., eds, Raven Press: New York, 1991; pp 11-19.
28. Doursout, M. F.; Wouters, P.; Kashimoto, S.; Hartley, C. J.; Rabinovitz, R.; Chelly, J. E., Measurement of cardiac function in conscious rats. Ultrasound Med Biol 2001, 27, (2), 195-202.
29. Hartley, C.; Lewis, R.; Hanley, H.; Cole, J. In Regional blood flow sensing in rats: a preliminary report, 11th International Conference on Medical and Biological Engineering, Ottawa, 1976;
30. Haywood, J. R.; Shaffer, R. A.; Fastenow, C.; Fink, G. D.; Brody, M. J., Regional blood flow measurement with pulsed Doppler flowmeter in conscious rat. Am J Physiol 1981, 241, (2), H273-8.
31. Keppel, G., Correction for multiple comparisons. In Design and Analysis, ed.; ‘Ed.’̂‘Eds.’ Prentice-Hall, Inc.,: Englewood Cliffs, N.J., 1982; ‘Vol.’ p̂pp 144-66.
32. Stasch, J. P.; Alonso-Alija, C.; Apeler, H.; Dembowsky, K.; Feurer, A.; Minuth, T.; Perzborn, E.; Schramm, M.; Straub, A., Pharmacological actions of a novel NO-independent guanylyl cyclase stimulator, BAY 41-8543: in vitro studies. Br J Pharmacol 2002, 135, (2), 333-43.
33. Okuda, K., Discovery of vitamin B12 in the liver and its absorption factor in the stomach: a historical review. J Gastroenterol Hepatol 1999, 14, (4), 301-8.
34. Stasch, J. P.; Dembowsky, K.; Perzborn, E.; Stahl, E.; Schramm, M., Cardiovascular actions of a novel NO-independent guanylyl cyclase stimulator, BAY 41-8543: in vivo studies. Br J Pharmacol 2002, 135, (2), 344-55.
35. Radomski, M. W.; Palmer, R. M.; Moncada, S., Endogenous nitric oxide inhibits human platelet adhesion to vascular endothelium. Lancet 1987, 2, (8567), 1057-8.
36. Kubes, P.; Suzuki, M.; Granger, D. N., Nitric oxide: an endogenous modulator of leukocyte adhesion. Proc Natl Acad Sci USA 1991, 88, (11), 4651-5.
37. Parker, J. O., Nitrate tolerance in angina pectoris. Cardiovasc Drugs Ther 1989, 2, (6), 823-9.
38. Becker, E. M.; Schmidt, P.; Schramm, M.; Schroder, H.; Walter, U.; Hoenicka, M.; Gerzer, R.; Stasch, J. P., The vasodilator-stimulated phosphoprotein (VASP): target of YC-1 and nitric oxide effects in human and rat platelets. J Cardiovasc Pharmacol 2000, 35, (3), 390-7.
39. Born; Gross, The aggregation of blood platelets. J. Physiol 1963, 168, 178-95.
40. Hobbs, A. J.; Moncada, S., Antiplatelet properties of a novel, non-NO-based soluble guanylate cyclase activator, BAY 41-2272. Vascul Pharmacol 2003, 40, (3), 149-54.
41. Sessler, J. L.; Seidel, D., Synthetic expanded porphyrin chemistry. Angew Chem Int Ed Engl 2003, 42, (42), 5134-75.
42. Ignarro, L. J.; Ballot, B.; Wood, K. S., Regulation of soluble guanylate cyclase activity by porphyrins and metalloporphyrins. J Biol Chem 1984, 259, (10), 6201-7.
43. Carr, H. S.; Tran, D.; Reynolds, M. F.; Burstyn, J. N.; Spiro, T. G., Activation of soluble guanylyl cyclase by four-coordinate metalloporphyrins: evidence for a role for porphyrin conformation. Biochemistry 2002, 41, (31), 10149-57.
44. Makino, R.; Matsuda, H.; Obayashi, E.; Shiro, Y.; Iizuka, T.; Hori, H., EPR characterization of axial bond in metal center of native and cobalt-substituted guanylate cyclase. J Biol Chem 1999, 274, (12), 7714-23.
45. Krautler, B., Vitamin B12: chemistry and biochemistry. Biochem Soc Trans 2005, 33, (Pt 4), 806-10.
46. Meier-Callahan, A. E.; Di Bilio, A. J.; Simkhovich, L.; Mahammed, A.; Goldberg, I.; Gray, H. B.; Gross, Z., Chromium corroles in four oxidation States. Inorg Chem 2001, 40, (26), 6788-93.
47. Robinson, R. R.; Doursout, M. F.; Chelly, J. E.; Powell, M. R.; Little, T. M.; Butler, B. D., Cardiovascular deconditioning and venous air embolism in simulated microgravity in the rat. Aviat Space Environ Med 1996, 67, (9), 835-40.

What is claimed is:

1. A method of activating soluble guanylyl cyclase (sGC) in a patient, comprising:
administering to the patient a composition comprising a corrin having the general formula (I) 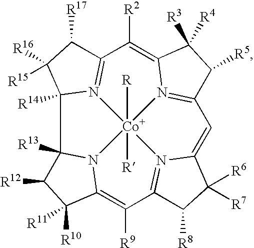
wherein R is selected from the group consisting of —CN, —N³⁻, —OH, —CₓH₂ₓ₊₁ wherein x is an integer from 1 to about 10, inclusive, and -deoxyadenosyl;
R¹ is selected from the group consisting of —CN, —N³⁻, —OH, —CₓH₂ₓ₊₁ wherein x is an integer from 1 to about 10, inclusive, -deoxyadenosyl, and dimethylbenzimidazolyl;
R² is selected from the group consisting of —H and —CₓH₂ₓ₊₁ wherein x is an integer from 1 to about 10, inclusive;
R³ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R⁴ is selected from the group consisting of —H and —CₓH₂ₓ₊₁ wherein x is an integer from 1 to about 10, inclusive;
R⁵ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R⁶ is selected from the group consisting of —H and —CₓH₂ₓ₊₁ wherein x is an integer from 1 to about 10, inclusive;
R⁷ is selected from the group consisting of —H and —CₓH₂ₓ₊₁ wherein x is an integer from 1 to about 10, inclusive;
R⁸ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R⁹ is selected from the group consisting of —H and —CₓH₂ₓ₊₁ wherein x is an integer from 1 to about 10, inclusive;
R¹⁰ is selected from the group consisting of —H and —CₓH₂ₓ₊₁ wherein x is an integer from 1 to about 10, inclusive;
R¹¹ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, —C_{y}H_{2y}CONH₂ wherein y is an integer from 1 to about 9, inclusive, —CH₂CH₂CONHCH₂CH(OH)CH₃, and —C_{y}H_{2y}CONHCH₂CHOP(OC₅O₃H₈)O₂ wherein y is an integer from 1 to about 9, inclusive, and (C₅O₃H₈) forms a ribose group;
R¹² is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R¹³ is selected from the group consisting of —H and —CₓH₂ₓ₊₁ wherein x is an integer from 1 to about 10, inclusive;
R¹⁴ is selected from the group consisting of —H and —CₓH₂ₓ₊₁ wherein x is an integer from 1 to about 10, inclusive;
R¹⁵ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R¹⁶ is selected from the group consisting of —H and —CₓH₂ₓ₊₁ wherein x is an integer from 1 to about 10, inclusive; and
R¹⁷ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
and a pharmaceutically-acceptable carrier.

2. The method of claim 1, wherein the corrin is selected from the group consisting of dicyanocobinamide (Factor B), cyanocobalamin (vitamin B₁₂), methylcobalamin (MeB₁₂), and 5-deoxyadenosylcobalamin (AdoB₁₂).

3. The method of claim 1, wherein the composition further comprises an NO-independent sGC activator.

4. The method of claim 3, wherein the NO-independent sGC activator is selected from the group consisting of BAY 41-2272, BAY 58-2667, and HMR1766.

5. The method of claim 3, wherein the NO-independent sGC activator is selected from the group consisting of YC-1 and S3448.

6. A composition, comprising:
a corrin having the general formula (I) 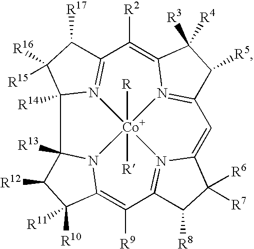
wherein R is selected from the group consisting of —CN, —N³⁻, —OH, —CₓH₂ₓ₊₁ wherein x is an integer from 1 to about 10, inclusive, and -deoxyadenosyl;
R¹ is selected from the group consisting of —CN, —N³⁻, —OH, —CₓH₂ₓ₊₁ wherein x is an integer from 1 to about 10, inclusive, -deoxyadenosyl, and dimethylbenzimidazolyl;
R² is selected from the group consisting of —H and —CₓH₂ₓ₊₁ wherein x is an integer from 1 to about 10, inclusive;
R³ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R⁴ is selected from the group consisting of —H and —CₓH₂ₓ₊₁ wherein x is an integer from 1 to about 10, inclusive;
R⁵ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R⁶ is selected from the group consisting of —H and —CₓH₂ₓ₊₁ wherein x is an integer from 1 to about 10, inclusive;
R⁷ is selected from the group consisting of —H and —CₓH₂ₓ₊₁ wherein x is an integer from 1 to about 10, inclusive;
R⁸ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R⁹ is selected from the group consisting of —H and —CₓH₂ₓ₊₁ wherein x is an integer from 1 to about 10, inclusive;
R¹⁰ is selected from the group consisting of —H and —CₓH₂ₓ₊₁ wherein x is an integer from 1 to about 10, inclusive;
R¹¹ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, —C_{y}H_{2y}CONH₂ wherein y is an integer from 1 to about 9, inclusive, —CH₂CH₂CONHCH₂CH(OH)CH₃, and —C_{y}H_{2y}CONHCH₂CHOP(OC₅O₃H₈)O₂ wherein y is an integer from 1 to about 9, inclusive, and (C₅O₃H₈) forms a ribose group;
R¹² is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R¹³ is selected from the group consisting of —H and —CₓH₂ₓ₊₁ wherein x is an integer from 1 to about 10, inclusive;
R¹⁴ is selected from the group consisting of —H and —CₓH₂ₓ₊₁ wherein x is an integer from 1 to about 10, inclusive;
R¹⁵ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
R¹⁶ is selected from the group consisting of —H and —CₓH₂ₓ₊₁ wherein x is an integer from 1 to about 10, inclusive; and
R¹⁷ is selected from the group consisting of —H, —CₓH₂ₓ₊₁ wherein x is an integer from 1 to about 10, inclusive, —C_{y}H_{2y}COOH and salts or esters thereof wherein y is an integer from 1 to about 9, inclusive, and —C_{y}H_{2y}CONH₂ wherein y is an integer from 1 to about 9, inclusive;
an NO-independent sGC activator selected from the group consisting of BAY 41-2272, BAY 58-2667, HMR1766, YC-1, and S3448; and
a pharmaceutically-acceptable carrier.

7. The composition of claim 6, wherein the corrin is selected from the group consisting of dicyanocobinamide (Factor B), cyanocobalamin (vitamin B₁₂), methylcobalamin (MeB₁₂), and 5-deoxyadenosylcobalamin (AdoB₁₂).
